SEQUENCE LISTING (4 sequences)

SEQ ID NO: 1 - DNA, 741 bp, Azoarcus communis
  CDS (1)..(741)
atgaatcagaaagtcgcactcgtcaccggcgccatgggtggcctgggtaccgctatctgccaggcgctggcaaaggacggaatgaaggtcgtggccaattgtctccccggctttccgcagaaggatgagtggctgggacggcagaaggagctcggcttcgatttcatcgctgccgaaggcgacgtatcggactatgactcctgtcgcgcgatggtggcgaagatcgagggcgaggtgggtgcgatcgatgtgctggtgaacaacgccgggatcacccgcgacaagttcttcccgaagatggaaaaggtgcagtgggatgcggtcatcaacaccaacctcaacagccttttcaacgtcactcaccacgtttcgccgaagatggcagaacggggctatggccgaatcatcaatatttcttcggtgaacggcgtcaagggccaggccggccagaccaactactcgactgccaaggcgggcgtgctcggcttcacgaaagcccttgccgcggaactggcgacgaaaggcgtgaccgtcaatgcgatcgcgccgggctatatcggcaccgagatggtgatggcgattcgcgaagacattcgccagggcatcatcgacagcgtcccgatgaagcgcctgggcaagccggaagaaatcggcgctctgtgctcctacctgtcttccgatctggccggttacgtgaccggcgcgacgatcaacatcaacggcggcctccacatgtgctga

SEQ ID NO: 2 - protein, 246 aa, Azoarcus communis
MNQKVALVTGAMGGLGTAICQALAKDGMKVVANCLPGFPQKDEWLGRQKELGFDFIAAEGDVSDYDSCRAMVAKIEGEVGAIDVLVNNAGITRDKFFPKMEKVQWDAVINTNLNSLFNVTHHVSPKMAERGYGRIINISSVNGVKGQAGQTNYSTAKAGVLGFTKALAAELATKGVTVNAIAPGYIGTEMVMAIREDIRQGIIDSVPMKRLGKPEEIGALCSYLSSDLAGYVTGATININGGLHMC

SEQ ID NO: 3 - DNA, 747 bp, Azoarcus communis
  CDS (1)..(747)
atgctgctcgaagggaaaaccgcgctggtgacgggtgccggcaacggcatcggccgcaccatcgcgctcacctacgccgccgaaggggcgaacgtcgtcgtttccgacatcagtgacgaatggggccgggaaacactcgccctgatcgaaggcaagggcggaaaagccgttttccaacacgccgacaccgcccaccccgaagaccatgacgagctgatcgccgcggccaaacgcgccttcggccgcctcgacattgcctgcaacaacgccggcatcagcggcgaattcacccctaccgcggaaacgaccgacgcccagtggcaacgagtcatcggcatcaacctgtcgggcgtgttctacggcgtgcgtgcgcagattcgcgccatgctcgaaaccggaggcggcgcgatcgtcaatatttcttccattgccgggcagatcggcatcgagggcatcacgccctacaccgccgccaagcacggcgtggtgggtctgacgaaaacggtcgcctgggaatatggcagcaagggcatccgcatcaattcggtcggtccggccttcatcaataccacgctggttcagaacgttcccctcgaaacacgccggcagctcgaacagatgcacgccctgcgccgcctaggcgaaacggaagaagtcgccaatctcgtcgcctggctgagcagcgacaaggccagcttcgtcaccggcagctattacgcggtcgacggcggctacctcgcacgatga

SEQ ID NO: 4 - protein, 248 aa, Azoarcus communis
MLLEGKTALVTGAGNGIGRTIALTYAAEGANVVVSDISDEWGRETLALIEGKGGKAVFQHADTAHPEDHDELIAAAKRAFGRLDIACNNAGISGEFTPTAETTDAQWQRVIGINLSGVFYGVRAQIRAMLETGGGAIVNISSIAGQIGIEGITPYTAAKHGVVGLTKTVAWEYGSKGIRINSVGPAFINTTLVQNVPLETRRQLEQMHALRRLGETEEVANLVAWLSSDKASFVTGSYYAVDGGYLAR

(19) United States
(12) Patent Application Publication
(10) Pub. No.: US 2012/0135476 A1
(43) Pub. Date: May 31, 2012

(54) PROCESS FOR PREPARING N-HETEROCYCLIC OPTICALLY ACTIVE ALCOHOLS

(75) Inventors: NINA SCHNEIDER, OFFENBURG (DE); MELANIE BONNEKESSEL, LUDWIGSHAFEN (DE); MICHAEL BREUER, DARMSTADT (DE); JÜRGEN DÄUWEL, HEIDELBERG (DE); KLAUS DITRICH, GONNHEIM (DE); ULRICH KARL, GRUNSTADT (DE); TOBIAS STÄB, FRANKENTHAL (DE)

(73) Assignee: BASF SE, LUDWIGSHAFEN (DE)

(21) Appl. No.: 13/303,559

(22) Filed: Nov. 23, 2011

Related U.S. Application Data
(60) Provisional application No. 61/416,776, filed on Nov. 24, 2010.

(51) Int. Cl. C12P 17/00

(52) U.S. Cl. 435/117

(57) ABSTRACT

Process for preparing N-heterocyclic optically active alcohols of the formula I

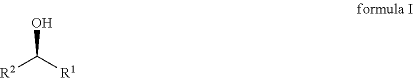

by reduction of the corresponding ketone, where the reduction is carried out using a dehydrogenase having the polypeptide sequence SEQ ID NO:2 or NO:4, or using a polypeptide sequence where, compared to SEQ ID NO:2 or NO:4, up to 25% of the amino acid residues are modified by deletion; insertion, substitution or a combination thereof.

The present invention relates to a process for preparing N-heterocyclic optically active alcohols using dehydrogenases.

### PRIOR ART

The action of dehydrogenases as biocatalysts is generally known [Chemico-Biological Interactions (2003) 143:247, Journal of Biological Chemistry (2002) 277:25677]. In particular the industrial use of this class of enzymes for preparing fine chemicals is documented [Tetrahedron (2004) 60:633, Trends Biotechnol (1999) 17:487]. Depending on the substrate, the known dehydrogenases differ in their activity and specificity. With a view to their stereoselectivity, a distinction is made between ‘Prelog’ and ‘anti-Prelog’ enzymes (Pure and Applied Chemistry, (1964), 9:119).

Thus, the biocatalysts described for preparing optically active phenylethanol derivatives are mainly those having ‘Prelog’ selectivity; enzymes having the opposite enantioselectivity are encountered less frequently, but are not unknown [Trends Biotechnol (1999) 17:487, J. Org. Chem. (1992) 57:1532]

### DESCRIPTION OF THE INVENTION

The present invention relates to a process for preparing optically active alcohols of the formula I

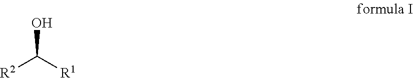

in which
   R¹ denotes alkyl groups which for their part may be mono- or polysubstituted by alkyl, halogen, SH, SR³, OH, OR³, NO₂, CN, CO, COOR³, NR³R⁴ or NR³R³R⁵⁺X⁻, where R³, R⁴ and R⁵ independently of one another are H or a lower alkyl or lower alkoxy radical and X⁻ is a counterion
   R² denotes N-containing heteroaryl groups which for their part may be mono- or polysubstituted by alkyl, halogen, SH, SR³, OH, OR³, NO₂, CN, CO, COOR³, NR³R⁴ or NR³R³R⁵⁺X⁻, where R³, R⁴ and R⁵ independently of one another are H or a lower alkyl or lower alkoxy radical and X⁻ is a counterion
   by reduction of the corresponding ketone, where the reduction is carried out using a dehydrogenase having the polypeptide sequence SEQ ID NO:2 or NO:4, or using a polypeptide sequence where, compared to SEQ ID NO:2 or NO:4, up to 25% of the amino acid residues are modified by deletion; insertion, substitution or a combination thereof.

A particularly good embodiment of the invention consists in a process for preparing optically active alcohols of the formula I in which R¹ is C1-C5-alkyl and R² is pyridinyl, in particular a 4-pyridinyl radical, where the radicals R1 and/or R2 are optionally monosubstituted by halogen.

The process according to the invention affords optically active alcohols having the (S)-configuration.

This process is also suitable for preparing optically active alcohols comprising N-containing non-aromatic heterocycles by hydrogenating the N-containing heteroaryl radical subsequent to the process described above.

The invention furthermore relates to a process for preparing optically active alcohols of the formula III,

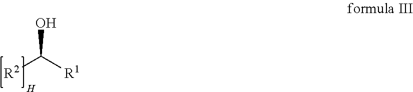

by carrying out the process according to claim 1 in a first step (a) and converting, in a second step (b), the optically active alcohol of the formula I obtained in (a) into III by hydrogenation.
In formula III, R¹ denotes alkyl groups which for their part may be mono- or polysubstituted by alkyl, halogen, SH, SR³, OH, OR³, NO₂, CN, CO, COOR³, NR³R⁴ or NR³R³R⁵⁺X⁻, where R³, R⁴ and R⁵ independently of one another are H or a lower alkyl or lower alkoxy radical and X⁻ is a counterion.
[R²]_{H} denotes saturated N-containing heterocycles which for their part may be mono- or polysubstituted by alkyl, halogen, SH, SR³, OH, OR³, NO₂, CN, CO, COOR³, NR³R⁴ or NR³R³R⁵⁺X⁻, where R³, R⁴ and R⁵ independently of one another are H or a lower alkyl or lower alkoxy radical and X⁻ is a counterion.

The hydrogenation in step (b) can be carried out using any method known to the skilled worker as being suitable for hydrogenating aromatics. Preference is given here to using hydrogenation catalysts comprising the elements Ru, Co, Rh, Ni, Pd or Pt (see Yang, P., Ed., The Chemistry of Nanostructured Materials, World Scientific Publishing: Singapore, (2003); pp. 329-357.)

Isolation and work-up of the reaction product obtained can be carried out using any customary method such as distillation, chromatography and crystallization. The reaction is usually followed by a distillation step, preferably a molecular distillation. The product can usually be obtained in high chemical purity, for example by crystallization.

If the optical purity of the alcohols obtained by the processes above is insufficient for certain areas of use, further purification, for example by fractional crystallization or diastereomer formation and separation is recommended.

A particularly preferred process for compounds of the formula III where [R²]_{H}=4-piperidinyl is the salt formation of this alcohol with an optically active acid, in particular with (R)-mandelic acid, and the crystallization of this salt. The alcohol can then be released again from the salts of the optically active acid by treatment with a base.

### GENERAL TERMS AND DEFINITIONS

Unless stated otherwise, the following general definitions apply:

“Alkyl” denotes straight-chain or branched alkyl radicals having 1 to 10, preferably 2-8, in particular 3-6, carbon atoms, in particular methyl, ethyl, iso- or n-propyl, n-, iso-, sec- or tert-butyl, n-pentyl or 2-methylbutyl, n-hexyl, 2-methylpentyl, 3-methylpentyl, 2-ethylbutyl, 2-ethylhexyl.

“Halogen” denotes fluorine, chlorine, bromine or iodine, in particular fluorine or chlorine.

“Lower alkyl” denotes straight-chain or branched alkyl radicals having 1 to 6 carbon atoms, such as methyl, ethyl, iso- or n-propyl, n-, iso-, sec- or tert-butyl, n-pentyl or 2-methylbutyl, n-hexyl, 2-methylpentyl, 3-methylpentyl, 2-ethylbutyl.

“N-containing heteroaryl” denotes a mono- or polycyclic, preferably mono- or bicyclic, optionally substituted heteroaromatic radical which carries at least one nitrogen atom as component of the aromatic system, in particular pyridinyl which is attached via any ring position. The ring size of the N-containing heteroaryl is preferably 5 or 6 members. In addition to the nitrogen atom which must be present, the N-containing heteroaryl may also carry further heteroatoms selected from the group consisting of N, O and S. These N-containing heteroaryl radicals may optionally carry 1, 2 or 3 identical or different substituents, for example halogen, lower alkyl, lower alkoxy according to the above definition or trifluoromethyl. The N-containing heteroaryl radical may be attached via any ring position of the N-containing heteroaryl radical to the other radicals of the compound of the formula I. In the case of pyridinyl as N-containing heteroaryl, the attachment is preferably as 4-pyridinyl.

“Enantioselectivity” in the context of the present invention means that the enantiomeric excess ee (in %) of one of the two possible enantiomers is at least 50%, preferably at least 80%, in particular at least 90% and especially at least 95%. The ee is calculated as follows:

ee(%)=enantiomer A−enantiomer B/(enantiomer A+enantiomer B)×100

### BIOCHEMICAL EMBODIMENTS

Particularly suitable dehydrogenases (EC 1.1.X.X) are especially NAD- or NADP-dependent dehydrogenases (E.C. 1.1.1.x), in particular alcohol dehydrogenases (E.C.1.1.1.1 or E.C.1.1.1.2) which bring about selective reduction of the ketone to the ‘Prelog’ alcohol. The dehydrogenase is preferably obtained from a microorganism, particularly preferably from a bacterium, a fungus, in particular a yeast, in each case deposited in collections of strains or obtainable from isolates of natural source, such as soil samples, biomass samples and the like or by de novo gene synthesis.

The dehydrogenase can be used in purified or partially purified form or in the form of the original microorganism or of a recombinant host organism which expresses the dehydrogenase. Processes for obtaining and purifying dehydrogenases from microorganisms are sufficiently well known to the skilled worker, e.g. from K. Nakamura & T. Matsuda, “Reduction of Ketones” in K. Drauz and H. Waldmann, Enzyme Catalysis in Organic Synthesis 2002, Vol. III, 991-1032, Wiley-VCH, Weinheim. Recombinant processes for generating dehydrogenases are likewise known, for example from W. Hummel, K. Abokitse, K. Drauz, C. Rollmann and H. Gröger, Adv. Synth. Catal. 2003, 345, No. 1+2, pp. 153-159.

Suitable bacteria are for example those of the orders of Burkholderiales, Hydrogenophilales, Methylophilales, Neisseriales, Nitrosomonadales, Procabacteriales or Rhodocyclales.

Particularly preferred dehydrogenases are those from the family the family of Rhodocyclaceae.

Especially preferred dehydrogenases are from the genera Azoarcus, Azonexus, Azospira, Azovibrio, Dechloromonas, Ferribacterium, Petrobacter, Propionivibrio, Quadricoccus, Rhodocyclus, Sterolibacterium, Thauera and Zoogloea.

Particularly preferred dehydrogenases are from species of the genera Azoarcus.

The reduction with the dehydrogenase normally takes place in the presence of a suitable cofactor (also referred to as cosubstrate). The cofactor normally used for reducing the ketone is NADH and/or NADPH. It is possible besides to employ dehydrogenases as cellular systems which intrinsically comprise cofactor, or alternative redox mediators can be added (A. Schmidt, F. Hollmann and B. Bühler “Oxidation of Alcohols” in K. Drauz and H. Waldmann, Enzyme Catalysis in Organic Synthesis 2002, Vol. III, 991-1032, Wiley-VCH, Weinheim).

The reduction with the dehydrogenase normally additionally takes place in the presence of a suitable reducing agent which regenerates the cofactor oxidized during the reduction. Examples of suitable reducing agents are sugars, especially the hexoses such as glucose, mannose, fructose, and/or oxidizable alcohols, especially ethanol, propanol, butanol, pentanol or isopropanol, and formate, phosphite or molecular hydrogen. To oxidize the reducing agent and, connected therewith, to regenerate the coenzyme it is possible to add a second dehydrogenase such as, for example, glucose dehydrogenase when glucose is used as reducing agent, phosphite dehydrogenase when phosphite is used as reducing agent or formate dehydrogenase when formate is used as reducing agent. This dehydrogenase can be employed as free or immobilized enzyme or in the form of free or immobilized cells. Preparation thereof is possible either separately or by coexpression in a (recombinant) dehydrogenase strain.

The dehydrogenases used according to the invention can be employed free or immobilized. An immobilized enzyme means an enzyme which is fixed to an inert carrier. Suitable carrier materials, and the enzymes immobilized thereon, are disclosed in EP-A-1149849, EP-A-1069183 and DE-A 100193773, and the references cited therein. The disclosure of these publications in this regard is incorporated in its entirety herein by reference. Suitable carrier materials include for example clays, clay minerals such as kaolinite, diatomaceous earth, perlite, silicon dioxide, aluminum oxide, sodium carbonate, calcium carbonate, cellulose powder, anion exchanger materials, synthetic polymers such as polystyrene, acrylic resins, phenol-formaldehyde resins, polyurethanes and polyolefins such as polyethylene and polypropylene. The carrier materials are normally employed in a finely divided particulate form to prepare the carrier-bound enzymes, with preference for porous forms. The particle size of the carrier material is normally not more than 5 mm, in particular not more than 2 mm (grading curve). It is possible analogously to choose a free or immobilized form on use of the dehydrogenase as whole-cell catalyst. Examples of carrier materials are Ca alginate and carrageenan. Both enzymes and cells can also be crosslinked directly with glutaraldehyde (crosslinking to give CLEAs). Corresponding and further immobilization methods are described for example in J. Lalonde and A. Margolin “Immobilization of Enzymes” in K. Drauz and H. Waldmann, Enzyme Catalysis in Organic Synthesis 2002, Vol. III, 991-1032, Wiley-VCH, Weinheim.

The reaction can be carried out in aqueous or nonaqueous reaction media or in 2-phase systems or (micro)emulsions. The aqueous reaction media are preferably buffered solutions which ordinarily have a pH of from 4 to 8, preferably from 5 to 8. The aqueous solvent may, besides water, additionally comprise at least one alcohol, e.g. ethanol or isopropanol, or dimethyl sulfoxide.

Nonaqueous reaction media mean reaction media which comprise less than 1% by weight, preferably less than 0.5% by weight, of water based on the total weight of the reaction medium. The reaction is preferably carried out in an organic solvent. Examples of suitable solvents are aliphatic hydrocarbons, preferably having 5 to 8 carbon atoms, such as pentane, cyclopentane, hexane, cyclohexane, heptane, octane or cyclooctane, halogenated aliphatic hydrocarbons, preferably having one or two carbon atoms, such as dichloromethane, chloroform, carbon tetrachloride, dichloroethane or tetrachloroethane, aromatic hydrocarbons, such as benzene, toluene, the xylenes, chlorobenzene or dichlorobenzene, aliphatic acyclic and cyclic ethers or alcohols, preferably having 4 to 8 carbon atoms, such as diethyl ether, methyl tert-butyl ether, ethyl tert-butyl ether, dipropyl ether, diisopropyl ether, dibutyl ether, tetrahydrofuran or esters such as ethyl acetate or n-butyl acetate or ketones such as methyl isobutyl ketone or dioxane or mixtures thereof.

The reduction with the dehydrogenase is carried out for example in an aqueous-organic, in particular aqueous, reaction medium.

The ketone to be reduced is preferably employed in a concentration of from 0.1 g/l to 500 g/l, particularly preferably from 1 g/l to 200 g/l, in the enzymatic reduction and can be fed in continuously or discontinuously.

The enzymatic reduction ordinarily takes place at a reaction temperature below the deactivation temperature of the dehydrogenase employed and preferably at −10° C. at least. It is particularly preferably in the range from 0 to 100° C., in particular from 15 to 60° C. and specifically from 20 to 40° C., e.g. at about 30° C.

A possible procedure is for example to mix the ketone with the dehydrogenase, the solvent and, if appropriate, the coenzymes, if appropriate a second dehydrogenase to regenerate the coenzyme and/or further reducing agents, thoroughly, e.g. by stirring or shaking. However, it is also possible to immobilize the dehydrogenase(s) in a reactor, for example in a column, and to pass a mixture comprising the ketone and, if appropriate, coenzymes and/or cosubstrates through the reactor. For this purpose, the mixture can be circulated through the reactor until the desired conversion is reached. In this case, the keto group of the ketone is reduced to an OH group, resulting in substantially one of the two enantiomers of the alcohol. The reduction is ordinarily managed until the conversion is at least 70%, particularly preferably at least 85% and especially at least 95%, based on the ketone present in the mixture. The progress of the reaction, i.e. the sequential reduction of the ketone, can in this case be followed by conventional methods such as gas chromatography or high-pressure liquid chromatography.

A first subject matter of the invention relates to functional phenylethanol dehydrogenase mutants derived from the phenylethanol dehydrogenase EbN1 from Azoarcus sp. having an amino acid sequence according to SEQ ID NO: 2.

The dehydrogenases employed in the process of the invention are particularly preferably alcohol dehydrogenases having the following properties:

Alcohol dehydrogenase from Azoarcus having an amino acid sequence shown in SEQ ID NO:2, and alcohol dehydrogenases having amino acid sequences in which up to 25%, preferably up to 15%, particularly preferably up to 10, especially up to 5%, of the amino acid residues are altered by comparison with SEQ ID NO:2 by deletion, insertion, substitution or a combination thereof.

The invention relates in particular to functional alcohol dehydrogenase mutants derived from the alcohol dehydrogenase EbN1 from Azoarcus sp. having an amino acid sequence according to SEQ ID NO: 2, where the mutant has at least one mutation in at least one sequence range selected from

(1) sequence range 142 to 153 (also referred to as loop 2) and
(2) sequence range 190 to 211 (also referred to as helix alpha FG1).

The invention relates in particular to functional alcohol dehydrogenase mutants which additionally have at least one further mutation in a further sequence range selected from

(3) sequence range 93 to 96 (also referred to as loop 1)
(4) sequence range 241 to 249 (C terminus)
(5) sequence range 138 to 141 (hydrophilic region binding pocket, also referred to as loop 2) and
(6) Cys61 and/or Cys 83

The invention furthermore relates to functional alcohol dehydrogenase mutants derived from the alcohol dehydrogenase EbN1 from Azoarcus sp. having an amino acid sequence according to SEQ ID NO: 2, where the mutant is selected from the mutants listed in Table 1. Particular mention may be made of mutants where at least one of the following radicals is mutated:

T192, L197, M200, F201, L204, M246, L139, T140, T142, L146, I148, Y151, C61, C83, L186, where the amino acid in question is replaced by any other natural amino acid.

Mutants according to the invention are selected in particular from mutants comprising at least one of the following mutations:

Y151X_{A}, where X_{A}=A, R, N, E, Q, G, H, I, L, M, T or V;
T192X_{B}, where X_{B}=A, E, G, I, P, S, W, V or L;

### Further Modifications of Dehydrogenases of the Invention:

The invention likewise comprises “functional equivalents” of the specifically disclosed enzymes having dehydrogenase activity and the use of these in the methods of the invention.

“Functional equivalents” or analogs of the specifically disclosed enzymes are, for the purposes of the present invention, polypeptides which differ from said enzymes but still retain the desired biological activity such as substrate specificity, for example. Thus, for example, “functional equivalents” mean enzymes which reduce the ketone to the corresponding ‘anti-Prelog’ alcohol and which have at least 20%, preferably 50%, particularly preferably 75%, very particularly preferably 90% of the activity of an enzyme comprising one of the amino acid sequences listed under Seq ID 2 or Seq ID 4. Moreover, functional equivalents are preferably stable between pH 4 and 10 and advantageously have a pH optimum of between pH 5 and 8 and a temperature optimum in the range from 20° C. to 80° C.

“Functional equivalents” mean according to the invention in particular also mutants which have in at least one sequence position of the abovementioned amino acid sequences an amino acid different from the specifically mentioned amino acids but which have nevertheless one of the abovementioned biological activities. “Functional equivalents” thus comprise the mutants obtainable by one or more amino acid additions, substitutions, deletions and/or inversions, it being possible for said modifications to occur in any sequence position as long as they result in a mutant having the property profile of the invention. In particular, functional equivalence also exists when the reactivity patterns between mutant and unmodified polypeptide correspond qualitatively, i.e. when identical substrates are converted at different rates, for example.

Further examples of suitable amino acid substitutions can be found in the table below:

| Original residue | Exemplary substitutions |
|---|---|
| Ala | Ser |
| Arg | Lys |
| Asn | Gln; His |
| Asp | Glu |
| Cys | Ser |
| Gln | Asn |
| Glu | Asp |
| Gly | Pro |
| His | Asn; Gln |
| Ile | Leu; Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

“Functional equivalents” in the above sense are also “precursors” of the polypeptides described and also “functional derivatives” and “salts” of said polypeptides.

In this context, “precursors” are natural or synthetic precursors of the polypeptides with or without the desired biological activity.

The term “salts” means not only salts of carboxyl groups, but also acid addition salts of amino groups of the protein molecules of the invention. Salts of carboxyl groups can be prepared in a manner known per se and comprise inorganic salts such as, for example, sodium, calcium, ammonium, iron and zinc salts, and also salts with organic bases such as, for example, amines, such as triethanolamine, arginine, lysine, piperidine, and the like. The invention likewise relates to acid addition salts such as, for example, salts with mineral acids such as hydrochloric acid or sulfuric acid and salts with organic acids such as acetic acid and oxalic acid.

“Functional derivatives” of polypeptides of the invention may likewise be prepared on functional amino acid side groups or the N- or C-terminal end thereof with the aid of known techniques. Derivatives of this kind comprise, for example, aliphatic esters of carboxylic acid groups, amides of carboxylic acid groups, obtainable by reaction with ammonia or with a primary or secondary amine; N-acyl derivatives of free amino groups, prepared by reaction with acyl groups; or O-acyl derivatives of free hydroxyl groups, prepared by reaction with acyl groups.

In the event of a possible protein glycosylation, “functional equivalents” of the invention comprise proteins of the above-specified type in deglycosylated or glycosylated form and also modified forms obtainable by altering the glycosylation pattern.

“Functional equivalents” also comprise, of course, polypeptides obtainable from other organisms and also naturally occurring variants. For example, regions of homologous sequences can be determined by sequence comparison, and equivalent enzymes can be established on the basis of the specific requirements of the invention.

“Functional equivalents” likewise comprise fragments, preferably individual domains or sequence motifs, of the polypeptides of the invention, which have the desired biological function, for example.

Moreover, “functional equivalents” are fusion proteins which have any of the abovementioned polypeptide sequences or functional equivalents derived therefrom and at least one other heterologous sequence functionally different therefrom in functional N- or C-terminal linkage (i.e. without substantial mutual functional impairment of the fusion protein moieties). Nonlimiting examples of heterologous sequences of this kind are signal peptides or enzymes, for example.

The invention also relates to “functional equivalents” which are homologs of the specifically disclosed proteins. These have at least 75%, in particular at least 85%, such as, for example, 90%, 95%, 97% or 99%, homology to any of the specifically disclosed amino acid sequences, calculated by the algorithm of Pearson and Lipman, Proc. Natl. Acad, Sci. (USA) 85(8), 1988, 2444-2448. Percentage homology of a homologous polypeptide of the invention means in particular percentage identity of the amino acid residues based on the total length of one of the amino acid sequences specifically described herein.

Homologs of the proteins or polypeptides of the invention may be generated by mutagenesis, for example by point mutation or truncation of the protein.

Homologs of the proteins of the invention may be identified by screening combinatorial libraries of mutants, such as truncation mutants, for example. For example, it is possible to generate a variegated library of protein variants by combinatorial mutagenesis at the nucleic acid level, for example by enzymatically ligating a mixture of synthetic oligonucleotides. There is a multiplicity of methods which can be used to prepare libraries of potential homologs from a degenerate oligonucleotide sequence. Chemical synthesis of a degenerate gene sequence may be carried out in a DNA synthesizer, and the synthetic gene can then be ligated into a suitable expression vector. The use of a degenerate set of genes makes it possible to provide, in one mixture, all sequences which encode the desired set of potential protein sequences. Methods for synthesizing degenerate oligonucleotides are known to the skilled worker (e.g. Narang, S. A. (1983) Tetrahedron 39:3; Itakura et al. (1984) Annu. Rev. Biochem. 53:323; Itakura et al., (1984) Science 198:1056; Ike et al. (1983) Nucleic Acids Res. 11:477).

The prior art discloses a plurality of techniques for screening gene products of combinatorial libraries which have been prepared by point mutations or truncation and for screening cDNA libraries for gene products with a selected property. These techniques can be adapted to the rapid screening of the gene libraries which have been generated by combinatorial mutagenesis of homologs of the invention. The most frequently used techniques for screening large gene libraries subjected to high-throughput analysis comprise cloning of the gene library into replicable expression vectors, transforming suitable cells with the resulting vector library and expressing the combinatorial genes under conditions under which detection of the desired activity facilitates isolation of the vector encoding the gene whose product has been detected. Recursive ensemble mutagenesis (REM), a technique which increases the frequency of functional mutants in the libraries, may be used in combination with the screening assays in order to identify homologs (Arkin and Yourvan (1992) PNAS 89:7811-7815; Delgrave et al. (1993) Protein Engineering 6(3):327-331).

### Further Embodiment of Coding Nucleic Acid Sequences of the Invention

The invention relates to the use of nucleic acid sequences (single- and double-stranded DNA and RNA sequences such as, for example, cDNA and mRNA) coding for an enzyme having the dehydrogenase activity of the invention. Preference is given to nucleic acid sequences coding, for example, for amino acid sequences according to Seq ID 2 or Seq ID 4 or characteristic partial sequences thereof, or comprising nucleic acid sequences according to Seq ID 1 or Seq ID 3 or characteristic partial sequences thereof.

All nucleic acid sequences mentioned herein can be prepared from the nucleotide building blocks in a manner known per se by chemical synthesis, such as, for example, by fragment condensation of individual overlapping, complementary nucleic acid building blocks of the double helix. Oligonucleotides may be chemically synthesized, for example, in a known manner, by the phosphoramidite method (Voet, Voet, 2nd Edition, Wiley Press New York, pages 896-897). Annealing synthetic oligonucleotides and filling in gaps with the aid of the Klenow fragment of DNA polymerase, and ligation reactions and also general cloning methods are described in Sambrook et al. (1989), Molecular Cloning: A laboratory manual, Cold Spring Harbor Laboratory Press.

The invention also relates to nucleic acid sequences (single- and double-stranded DNA and RNA sequences such as cDNA and mRNA, for example) encoding any of the above polypeptides and their functional equivalents which are obtainable, for example, by using artificial nucleotide analogs.

The invention relates to both isolated nucleic acid molecules coding for polypeptides or proteins of the invention or for biologically active segments thereof and nucleic acid fragments which may be used, for example, for use as hybridization probes or primers for identifying or amplifying coding nucleic acids of the invention.

The nucleic acid molecules of the invention may moreover comprise untranslated sequences of the 3′- and/or 5′-end of the coding region of the gene.

The invention furthermore comprises the nucleic acid molecules complementary to the specifically described nucleotide sequences or a section of said nucleic acid molecules.

The nucleotide sequences of the invention make possible the generation of probes and primers which can be used for identifying and/or cloning homologous sequences in other cell types and organisms. Such probes and primers usually comprise a nucleotide sequence region which hybridizes under “stringent” conditions (see hereinbelow) to at least about 12, preferably at least about 25, such as, for example, about 40, 50 or 75, consecutive nucleotides of a sense strand of a nucleic acid sequence of the invention or of a corresponding antisense strand.

An “isolated” nucleic acid molecule is removed from other nucleic acid molecules present in the natural source of the nucleic acid and may, in addition, be essentially free of other cellular materials or culture medium, if produced by recombinant techniques, or free of chemical precursors or other chemicals, if chemically synthesized.

A nucleic acid molecule of the invention may be isolated by means of standard techniques of molecular biology and the sequence information provided according to the invention. For example, cDNA can be isolated from a suitable cDNA library by using any of the specifically disclosed complete sequences or a section thereof as hybridization probe and standard hybridization techniques (as described, for example, in Sambrook, J., Fritsch, E. F. and Maniatis, T. Molecular Cloning: A Laboratory Manual. 2nd Ed., Cold Spring Harbor Laboratory, Cold Spring Harbor Laboratory Press, Cold Spring Harbor, N.Y., 1989). In addition, a nucleic acid molecule comprising any of the disclosed sequences or a section thereof can be isolated by polymerase chain reaction using the oligonucleotide primers generated on the basis of said sequence. The nucleic acid amplified in this way may be cloned into a suitable vector and characterized by DNA sequence analysis. The oligonucleotides of the invention may furthermore be prepared by standard methods of synthesis, for example using an automatic DNA synthesizer.

The nucleic acid sequences of the invention can be identified and isolated in principle from all organisms. Advantageously, the nucleic acid sequences of the invention or homologs thereof can be isolated from fungi, yeasts, Archaea or bacteria. Gram-negative and gram-positive bacteria may be mentioned as bacteria. Preferably, the nucleic acids according to the invention are isolated from gram-negative bacteria, advantageously from a-proteobacteria, β-proteobacteria or γ-proteobacteria, particularly preferably from bacteria of the orders Burkholderiales, Hydrogenophilales, Methylophilales, Neisseriales, Nitrosomonadales, Procabacteriales or Rhodocyclales, very particularly preferably from bacteria of the family of the Rhodocyclaceae, especially preferably from the genus Azoarcus, most preferably from the species Azoarcus anaerobius, Azoarcus buckelii, Azoarcus communis, Azoarcus evansii, Azoarcus indigens, Azoarcus toluclasticus, Azoarcus tolulyticus, Azoarcus toluvorans, Azoarcus sp., Azoarcus sp. 22Lin, Azoarcus sp. BH72, Azoarcus sp. CC-11, Azoarcus sp. CIB, Azoarcus sp. CR23, Azoarcus sp. EB1, Azoarcus sp. EbN1, Azoarcus sp. FL05, Azoarcus sp. HA, Azoarcus sp. H×N1, Azoarcus sp. mXyN1, Azoarcus sp. PbN1, Azoarcus sp. PH002, Azoarcus sp. T and Azoarcus sp. ToN1.

Particular preference is given to using dehydrogenases from Azoarcus sp EbN1.

Nucleic acid sequences of the invention can be isolated from other organisms, for example via genomic or cDNA libraries, by using, for example, common hybridization methods or the PCR technique. These DNA sequences hybridize with the sequences of the invention under standard conditions. For hybridization, it is advantageous to use short oligonucleotides of the conserved regions, for example of the active site, which can be identified via comparisons with a dehydrogenase of the invention in a manner known to the skilled worker. However, it is also possible to use longer fragments of the nucleic acids of the present invention or the complete sequences for the hybridization. Said standard conditions vary depending on the nucleic acid used (oligonucleotide, longer fragment or complete sequence) or depending on the type of nucleic acids, DNA or RNA, being used for hybridization. Thus, for example, the melting temperatures of DNA:DNA hybrids are approx. 10° C. lower than those of DNA:RNA hybrids of the same length.

Standard conditions mean, for example, depending on the nucleic acid, temperatures between 42 and 58° C. in an aqueous buffer solution at a concentration of between 0.1 and 5×SSC (1×SSC=0.15 M NaCl, 15 mM sodium citrate, pH 7.2) or additionally in the presence of 50% formamide, for example 42° C. in 5×SSC, 50% formamide, The hybridization conditions for DNA:DNA hybrids are advantageously 0.1×SSC and temperatures between about 20° C. and 45° C., preferably between about 30° C. and 45° C. The hybridization conditions for DNA:RNA hybrids are advantageously 0.1×SSC and temperatures between about 30° C. and 55° C., preferably between about 45° C. and 55° C. These hybridization temperatures indicated are melting temperatures calculated by way of example for a nucleic acid of approx. 100 nucleotides in length and having a G+C content of 50% in the absence of formamide. The experimental conditions for DNA hybridization are described in relevant genetics textbooks, such as, for example, Sambrook et al., “Molecular Cloning”, Cold Spring Harbor Laboratory, 1989, and can be calculated using formulae known to the skilled worker, for example as a function of the length of the nucleic acids, the type of hybrid or the G+C content. Further information on hybridization can be found by the skilled worker in the following textbooks: Ausubel et al. (eds), 1985, Current Protocols in Molecular Biology, John Wiley & Sons, New York; Hames and Higgins (eds), 1985, Nucleic Acids Hybridization: A Practical Approach, IRL Press at Oxford University Press, Oxford; Brown (ed), 1991, Essential Molecular Biology: A Practical Approach, IRL Press at Oxford University Press, Oxford.

The invention also relates to derivatives of the specifically disclosed or derivable nucleic acid sequences.

Thus it is possible for further nucleic acid sequences of the invention to be derived from Seq ID 1 or Seq ID 3 and to differ therefrom by addition, substitution, insertion or deletion of one or more nucleotides, while still coding for polypeptides with the desired property profile.

The invention also comprises those nucleic acid sequences which comprise “silent mutations” or which are modified, in comparison with a specifically mentioned sequence, according to the codon usage of a specific source organism or host organism, and also naturally occurring variants thereof, such as splice variants or allelic variants, for example.

The invention also relates to sequences obtainable by conservative nucleotide substitutions (i.e. the amino acid in question is replaced with an amino acid of the same charge, size, polarity and/or solubility).

The invention also relates to the molecules derived from the specifically disclosed nucleic acids by way of sequence polymorphisms. These genetic polymorphisms may exist between individuals within a population, owing to natural variations. These natural variations usually cause a variance of from 1 to 5% in the nucleotide sequence of a gene.

Derivatives of an inventive nucleic acid sequence mean, for example, allelic variants having at least 40% homology at the deduced amino acid level, preferably at least 60% homology, very particularly preferably at least 80, 85, 90, 93, 95 or 98% homology, over the entire sequence region (with regard to homology at the amino acid level, reference may be made to the above comments regarding polypeptides). Advantageously, the homologies may be higher across parts of the sequence.

Furthermore, derivatives also mean homologs of the nucleic acid sequences of the invention, for example fungal or bacterial homologs, truncated sequences, single-stranded DNA or RNA of the coding or noncoding DNA sequence. Thus, for example, homologs at the DNA level are at least 40%, preferably at least 60%, particularly preferably at least 70%, very particularly preferably at least 80%, homologous over the entire DNA region.

Moreover, derivatives mean, for example, fusions with promoters. Said promoters which are located upstream of the nucleotide sequences indicated may have been modified by one or more nucleotide substitutions, insertions, inversions and/or deletions, without adversely affecting the functionality or efficacy of said promoters, however. Furthermore, the efficacy of said promoters may be increased by modifying their sequence, or said promoters may be replaced completely with more efficient promoters, including those of organisms of different species.

Derivatives also mean variants whose nucleotide sequence in the region of from −1 to −1000 bases upstream of the start codon or from 0 to 1000 bases downstream of the stop codon has been altered so as to modify, preferably increase, gene expression and/or protein expression.

Furthermore, the invention also comprises nucleic acid sequences which hybridize with the abovementioned coding sequences under “stringent conditions”. These polynucleotides can be identified when screening genomic or cDNA libraries and, if appropriate, be amplified therefrom by means of PCR using suitable primers and subsequently isolated using suitable probes, for example. In addition, polynucleotides of the invention may also be synthesized chemically. This property means the ability of a poly- or oligonucleotide to bind under stringent conditions to a virtually complementary sequence, while there are no nonspecific bindings between noncomplementary partners under these conditions. For this purpose, the sequences should be 70-100%, preferably 90-100%, complementary. The property of complementary sequences of being able to bind specifically to one another is exploited, for example, in the Northern or Southern blot technique or for primer binding in PCR or RT-PCR. For this purpose, oligonucleotides from a length of 30 base pairs are customarily used. Stringent conditions mean, for example in the Northern blot technique, using a washing solution, for example 0.1×SSC buffer containing 0.1% SDS (20×SSC: 3M NaCl, 0.3M sodium citrate, pH 7.0) at a temperature of 50-70° C., preferably 60-65° C., for eluting nonspecifically hybridized cDNA probes or oligonucleotides. In the process, only highly complementary nucleic acids remain bound to one another, as mentioned above. Setting of stringent conditions is known to the skilled worker and described, for example, in Ausubel et al., Current Protocols in Molecular Biology, John Wiley & Sons, N.Y. (1989), 6.3.1-6.3.6.

### Embodiments of Constructs of the Invention

The invention moreover relates to expression constructs comprising, under the genetic control of regulatory nucleic acid sequences, a nucleic acid sequence coding for a polypeptide of the invention; and also to vectors comprising at least one of said expression constructs.

Such constructs of the invention preferably comprise a promoter 5′ upstream of the particular coding sequence and a terminator sequence 3′ downstream and also, if appropriate, further common regulatory elements, in each case operatively linked to the coding sequence.

An “operative linkage” means the sequential arrangement of promoter, coding sequence, terminator and, if appropriate, further regulatory elements in such a way that each of said regulatory elements is able to carry out its function in expression of the coding sequence as required. Examples of operatively linkable sequences are targeting sequences and also enhancers, polyadenylation signals, and the like. Other regulatory elements comprise selectable markers, amplification signals, origins of replications, and the like. Suitable regulatory sequences are described, for example, in Goeddel, Gene Expression Technology: Methods in Enzymology 185, Academic Press, San Diego, Calif. (1990).

A nucleic acid construct of the invention means in particular those where the gene for a dehydrogenase of the invention has been operatively or functionally linked to one or more regulatory signals for controlling, for example increasing, gene expression.

In addition to these regulatory sequences, the natural regulation of said sequences may still be present upstream of the actual structural genes and, if appropriate, may have been genetically modified so that natural regulation has been switched off and expression of the genes has been increased. However, construction of the nucleic acid construct may also be simpler, i.e. no additional regulatory signals have been inserted upstream of the coding sequence, and the natural promoter together with its regulation has not been removed. Instead, the natural regulatory sequence has been mutated in such a way that regulation no longer takes place and gene expression is increased.

A preferred nucleic acid construct advantageously also comprises one or more of the already mentioned enhancer sequences, functionally linked to the promoter, which enable expression of the nucleic acid sequence to be increased. Additional advantageous sequences such as further regulatory elements or terminators may also be inserted at the 3′ end of the DNA sequences. One or more copies of the nucleic acids of the invention may be comprised in the construct. The construct may also comprise other markers such as resistances to antibiotics or auxotrophy-complementing genes, if appropriate, for selection for the construct.

Advantageous regulatory sequences for the process of the invention are present, for example, in promoters such as cos, tac, trp, tet, trp-tet, lpp, lac, lpp-lac, lacl^{q}, T7, T5, T3, gal, trc, ara, rhaP (rhaP_{BAD})SP6, lambda-P_{R} or in the lambda-P_{L} promoter, which are advantageously used in Gram-negative bacteria. Other advantageous regulatory sequences are comprised, for example, in the Gram-positive promoters amy and SPO2, in the yeast or fungal promoters ADC1, MFalpha, AC, P-60, CYC1, GAPDH, TEF, rp28, ADH. In this connection, the promoters of pyruvate decarboxylase and of methanol oxidase, for example from Hansenula, are also advantageous. It is also possible to use artificial promoters for regulation.

The nucleic acid construct is advantageously expressed in a host organism by inserting it into a vector such as a plasmid or a phage, for example, which makes possible optimal expression of the genes in the host. Vectors mean, apart from plasmids and phages, also any other vectors known to the skilled worker, i.e., for example, viruses such as SV40, CMV, Baculovirus and Adenovirus, transposons, IS elements, phasmids, cosmids, and linear or circular DNA. These vectors can be replicated autonomously in the host organism or chromosomally. These vectors constitute a further embodiment of the invention. Suitable plasmids are, for example, in E. coli pLG338, pACYC184, pBR322, pUC18, pUC19, pKC30, pRep4, pHS1, pKK223-3, pDHE19.2, pHS2, pPLc236, pMBL24, pLG200, pUR290, pIN-III¹¹³-B1, lgt11 or pBdCl, in Streptomyces pIJ101, pIJ364, pIJ702 or pIJ361, in Bacillus pUB110, pC194 or pBD214, in Corynebacterium pSA77 or pAJ667, in fungi pALS1, pIL2 or pBB116, in yeasts 2alphaM, pAG-1, YEp6, YEp13 or pEMBLYe23 or in plants pLGV23, pGHlac⁺, pBIN19, pAK2004 or pDH51. The plasmids mentioned are a small selection of possible plasmids. Other plasmids are well known to the skilled worker and can be found, for example, in the book Cloning Vectors (Eds. Pouwels P. H. et al. Elsevier, Amsterdam-New York-Oxford, 1985, ISBN 0 444 904018).

For expression of the further genes which are comprised, the nucleic acid construct advantageously additionally comprises 3′- and/or 5′-terminal regulatory sequences for enhancing expression, which are selected for optimal expression depending on the host organism and the gene or genes selected.

These regulatory sequences are intended to make possible specific expression of the genes and protein expression. Depending on the host organism, this may mean, for example, that the gene is expressed or overexpressed only after induction or that it is immediately expressed and/or overexpressed.

The regulatory sequences or factors may preferably have a positive effect on, and thus increase, gene expression of the genes introduced. Thus, the regulatory elements can advantageously be enhanced at the transcriptional level by using strong transcription signals such as promoters and/or enhancers. However, in addition it is also possible to enhance translation by improving, for example, stability of the mRNA.

In a further embodiment of the vector, the vector comprising the nucleic acid construct of the invention or the nucleic acid of the invention may advantageously also be introduced into the microorganisms in the form of a linear DNA and integrated via heterologous or homologous recombination into the genome of the host organism. This linear DNA may consist of a linearized vector such as a plasmid or only of the nucleic acid construct or the nucleic acid of the invention.

It is advantageous for optimal expression of heterologous genes in organisms to modify the nucleic acid sequences according to the specific codon usage used in the organism. The codon usage can be readily determined on the basis of computer evaluations of other, known genes of the organism in question.

An expression cassette of the invention is prepared by fusing a suitable promoter to a suitable coding nucleotide sequence and a terminator signal or polyadenylation signal. For this purpose, conventional recombination and cloning techniques are used, as are described, for example, in T. Maniatis, E. F. Fritsch and J. Sambrook, Molecular Cloning: A Laboratory Manual, Cold Spring Harbor Laboratory, Cold Spring Harbor, N.Y. (1989) and in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley Interscience (1987).

The recombinant nucleic acid construct or gene construct is expressed in a suitable host organism by inserting it advantageously into a host-specific vector which makes optimal expression of the genes in the host possible. Vectors are well known to the skilled worker and can be found, for example, in “Cloning Vectors” (Pouwels P. H. et al., eds., Elsevier, Amsterdam-New York-Oxford, 1985).

Host Organisms which are Usable According to the Invention

It is possible with the aid of the vectors or constructs of the invention to prepare recombinant microorganisms which are transformed, for example, with at least one vector of the invention and can be used for producing the polypeptides of the invention. The above-described recombinant constructs of the invention are advantageously introduced into a suitable host system and expressed. In this context, preference is given to using cloning and transfection methods familiar to the skilled worker, such as, for example, coprecipitation, protoplast fusion, electroporation, retroviral transfection and the like, in order to express the nucleic acids mentioned in the particular expression system. Suitable systems are described, for example, in Current Protocols in Molecular Biology, F. Ausubel et al., Eds., Wiley Interscience, New York 1997, or Sambrook et al. Molecular Cloning: A Laboratory Manual. 2nd Ed., Cold Spring Harbor Laboratory, Cold Spring Harbor Laboratory Press, Cold Spring Harbor, N.Y., 1989.

According to the invention, it is also possible to prepare microorganisms by homologous recombination. For this purpose, a vector is prepared which comprises at least one section of a gene of the invention or of a coding sequence, into which at least one amino acid deletion, addition or substitution has been introduced, if appropriate, in order to modify, for example to functionally disrupt, the sequence of the invention (“knockout” vector). The sequence introduced may also be, for example, a homolog from a related microorganism or may have been derived from a mammalian, yeast or insect source. Alternatively, the vector used for homologous recombination may be designed in such a way that the endogenous gene is mutated or otherwise modified upon homologous recombination, while still encoding the functional protein (for example, the upstream regulatory region may have been modified in such a way that this causes modified expression of the endogenous protein). The modified section of the gene of the invention is present in the homologous recombination vector. The construction of suitable vectors for homologous recombination is described, for example, in Thomas, K. R. and Capecchi, M. R. (1987) Cell 51:503.

Suitable recombinant host organisms for the nucleic acid of the invention or the nucleic acid construct are, in principle, all prokaryotic or eukaryotic organisms. Host organisms which are used advantageously are microorganisms such as bacteria, fungi or yeasts. Advantageously used are Gram-positive or Gram-negative bacteria, preferably bacteria of the families Enterobacteriaceae, Pseudomonadaceae, Rhizobiaceae, Streptomycetaceae or Nocardiaceae, particularly preferably bacteria of the genera Escherichia, Pseudomonas, Streptomyces, Nocardia, Burkholderia, Salmonella, Agrobacterium or Rhodococcus. Very particular preference is given to the genus and species Escherichia coli. In addition, further advantageous bacteria can be found in the group of a-proteobacteria, β-proteobacteria or γ-proteobacteria.

The host organism, or host organisms, according to the invention comprise at least one of the nucleic acid sequences, nucleic acid constructs or vectors which are described in the present invention and which code for an enzyme having inventive dehydrogenase activity.

Depending on the host organism, the organisms used in the process of the invention are cultured or grown in a manner known to the skilled worker. Microorganisms are usually grown in a liquid medium comprising a carbon source, usually in the form of sugars, a nitrogen source, usually in the form of organic nitrogen sources such as yeast extract or of salts such as ammonium sulfate, trace elements such as salts of iron, manganese, magnesium, and, if appropriate, vitamins, at temperatures between 0° C. and 100° C., preferably between 10° C. and 60° C., while passing in oxygen. The pH of the nutrient liquid may be kept constant there, i.e. regulated or not regulated during cultivation. Cultivation may be batchwise, semibatchwise or continuous. Nutrients may be introduced at the start of the fermentation or fed in semicontinuously or continuously. The ketone may be added directly to the cultivation or, advantageously, after cultivation. The enzymes may be isolated from the organisms by the method described in the examples or used as crude extract for the reaction.

### Recombinant Production of the Polypeptides of the Invention

The invention furthermore relates to methods for recombinant production of polypeptides of the invention or of functional, biologically active fragments thereof, in which method a polypeptide-producing microorganism is cultured, expression of said polypeptides is induced if appropriate, and the latter are isolated from the culture. If desired, the polypeptides may also be produced on the industrial scale in this manner.

The recombinant microorganism can be cultured and fermented by known methods. For example, bacteria may be propagated in TB or LB medium and at a temperature of from 20 to 40° C. and pH 6 to 9. Suitable culturing conditions are described in detail, for example, in T. Maniatis, E. F. Fritsch and J. Sambrook, Molecular Cloning: A Laboratory Manual, Cold Spring Harbor Laboratory, Cold Spring Harbor, N.Y. (1989).

Unless the polypeptides are secreted into the culture medium, the cells are then disrupted and the product is obtained from the lysate by known protein isolation methods. The cells can be disrupted either by high-frequency ultrasound, by high pressure, for example in a French press, by osmolysis, by the action of detergents, lytic enzymes or organic solvents, by homogenizers or by combining two or more of the methods listed.

The polypeptides can be purified using known chromatographic methods such as molecular sieve chromatography (gel filtration), for example Q-Sepharose chromatography, ion exchange chromatography and hydrophobic chromatography, and also by other customary methods such as ultrafiltration, crystallization, salting out, dialysis and native gel electrophoresis. Suitable methods are described, for example, in Cooper, F. G., Biochemische Arbeitsmethoden [The Tools of Biochemistry], Verlag Walter de Gruyter, Berlin, New York or in Scopes, R., Protein Purification, Springer Verlag, New York, Heidelberg, Berlin.

It may be advantageous to isolate the recombinant protein by using vector systems or oligonucleotides which extend the cDNA by particular nucleotide sequences and thus code for modified polypeptides or fusion proteins which simplify purification, for example. Suitable modifications of this kind are, for example, “tags” acting as anchors, such as, for example, the modification known as hexa-histidine anchor, or epitopes which can be recognized as antigens by antibodies (described, for example, in Harlow, E. and Lane, D., 1988, Antibodies: A Laboratory Manual. Cold Spring Harbor (N.Y.) Press). These anchors can be used for attaching the proteins to a solid support such as, for example, a polymer matrix which may be packed, for example, in a chromatography column or on a microliter plate or on any other support.

At the same time, these anchors may also be used for identifying the proteins. Moreover, customary labels such as fluorescent dyes, enzyme labels which, after reaction with a substrate, form a detectable reaction product or radiolabels may be used, alone or in combination with said anchors, for identifying the proteins in order to derivatize said proteins.

### Further Embodiments of Carrying Out the Enzymatic Reduction Process of the Invention

In the process of the invention, the dehydrogenases can be used as free or immobilized enzyme or as a catalyst which is still present in the recombinant production organism.

The process of the invention is advantageously carried out at a temperature between 0° C. and 95° C., preferably between 10° C. and 85° C., particularly preferably between 15° C. and 75° C.

The pH in the process of the invention is advantageously maintained between pH 4 and 12, preferably between pH 4.5 and 9, particularly preferably between pH 5 and 8.

In the process of the invention, enantiomerically pure or chiral products mean enantiomers which show enrichment of one enantiomer. The process preferably achieves enantiomeric purities of at least 70% ee, preferably of at least 80% ee, particularly preferably of at least 90% ee, very particularly preferably of at least 98% ee.

It is possible to use for the process of the invention growing cells which comprise the nucleic acids, nucleic acid constructs or vectors of the invention. It is also possible to use resting or disrupted cells. Disrupted cells mean, for example, cells which have been made permeable by treatment with, for example, solvents, or cells which have been ruptured by an enzyme treatment, by a mechanical treatment (for example French Press or ultrasound) or by another method. The crude extracts obtained in this way are advantageously suitable for the process of the invention. Purified or partially purified enzymes may also be used for the process. Likewise suitable are immobilized microorganisms or enzymes which can advantageously be utilized in the reaction.

The process of the invention can be carried out batchwise, semibatchwise or continuously.

The process may advantageously be carried out in bioreactors as described, for example, in Biotechnology, Volume 3, 2nd Edition, Rehm et al. Eds., (1993), in particular Chapter II.

The following examples are intended to illustrate the invention but without restricting it.

### Experimental Section

### Example 1

### Cloning of the ALCOHOL Dehydrogenase EbN2 from Azoarcus sp. EbN1

The sequence of the EbN2 dehydrogenase gene from Azoarcus sp. EbN1 is deposited in databases (SEQ ID NO:1, [Genbank ID 56475432, Region: 2797788.2798528]). Oligonucleotides were derived from the nucleic acid sequence of the gene and were used to amplify by known methods the gene from genomic DNA of Azoarcus sp. EbN1. The resulting sequence corresponds to the published sequence.

### PCR Conditions:

2 μl of 10*Pfu ultra buffer (Stratagene)
100 ng of primer #1
100 ng of primer #2
1 μl of dNTP (10 mM each)
ca. 30 ng of chromosomal DNA from Azoarcus sp. EbN1
1 U of Pfu ultra DNA polymerase
ad 20 μl H₂O

### Temperature Program:

| | | |
|---|---|---|
| 5 min, 94° C., | | |
| 60 sec, 50° C., | | |
| 2 min, 72° C., | | |
| 60 sec, 94° C., | | |
| 10 min, 72° C., | {close oversize brace} | (35 cycles) |
| cooling to 10° C. | | |

The PCR product (approx. 751 bp) was digested with the restriction endonucleases NdeI and BamHI and cloned into correspondingly digested pDHE19.2 vector (DE19848129). The ligation mixtures were transformed into E. coli XL1 Blue (Stratagene).

The resulting plasmid pDHE-PDH-L was transformed into the strain E. coli TG10 pAgro4 pHSG575 (TG10: an RhaA⁻ derivative of E. coli TG1(Stratagene); pAgro4: Takeshita, S; Sato, M; Toba, M; Masahashi, W; Hashimoto-Gotoh, T (1987) Gene 61, 63-74; pHSG575: T. Tomoyasu et al (2001), Mol. Microbiol. 40(2), 397-413).

The recombinant E. coli are referred to as LU 13151.

### Example 2

### Cloning of the Alcohol Dehydrogenase ChnA from Azoarcus sp. EbN1

The sequence of the dehydrogenase gene ChnA from Azoarcus sp. EbN1 is deposited in databases ([Genbank ID 56475432, Region: (complement) 192247 . . . 192993]). Oligonucleotides were derived from the nucleic acid sequence of the gene and were used to amplify by known methods the gene from genomic DNA of Azoarcus sp. EbN1. The resulting sequence corresponds to the published sequence.

### PCR Conditions:

2 μl of 10*Pfu ultra buffer (Stratagene)
100 ng of primer #3
100 ng of primer #4
1 μl of dNTP (10 mM each)
ca. 30 ng of chromosomal DNA from Azoarcus sp. EbN1
1 U of Pfu ultra DNA polymerase

### ad 20 μl H₂O

### Temperature Program:

| | | |
|---|---|---|
| 5 min, 94° C., | | |
| 60 sec, 50° C., | | |
| 2 min, 72° C., | | |
| 60 sec, 94° C., | | |
| 10 min, 72° C., | {close oversize brace} | (35 cycles) |
| cooling to 10° C. | | |

The PCR product (approx. 743 bp) was digested with the restriction endonucleases NdeI and BglII and cloned into a pDHE19.2 vector (DE19848129) restricted with NdeI and Bam-HI. The ligation mixtures were transformed into E. coli XL1 Blue (Stratagene).

The resulting plasmid pDHE-PDH-L was transformed into the strain E. coli TG10 pAgro4 pHSG575 (TG10: an RhaA⁻ derivative of E. coli TG1(Stratagene); pAgro4: Takeshita, S; Sato, M; Toba, M; Masahashi, W; Hashimoto-Gotoh, T (1987) Gene 61, 63-74; pHSG575: T. Tomoyasu et al (2001), Mol. Microbiol. 40(2), 397-413).

The recombinant E. coli are referred to as LU 13283.

### Example 3

### Provision of Recombinant ‘Anti-Prelog’ Dehydrogenases

LU 13151 or LU 13283 were grown in 20 ml of LB-Amp/Spec/Cm (100 μg/l ampicillin;

100 μg/l spectinomycin; 20 μg/l chloramphenicol), 0.1 mM IPTG, 0.5 g/l rhamnose in 100 ml Erlenmeyer flasks (baffles) at 37° C. for 18 h (alternatively, a preculture of the same antibiotics concentration, but without IPTG and rhamnose, may be prepared first. This is incubated at 37° C. for 5 h and then used to inoculate the main culture at 1%), centrifuged at 5000*g/10 min, washed once with 10 mM TRIS*HCl, pH 7.0, and resuspended in 2 ml of the same buffer.

Cell-free crude protein extract was prepared by disrupting LU 13151 or LU 13283 cell paste using 0.7 ml glass beads (d=0.5 mm) in a vibratory mill (3×5 min with intermediate cooling on ice).

### Example 4

### Determination of the Activity of the Recombinant ‘Anti-Prelog’ Dehydrogenases from Azoarcus sp. EbN1

6 transformants in each case were grown in 20 ml of LB Amp/Spec/Cm (100 μg/l amp; 100 mg/l spec; 20 μg/lcm) 0.1 mM IPTG 0.5 g/l rhamnose in 100 ml Erlenmeyer flasks (baffles) at 37° C. for 18 h, centrifuged at 5000*g/10 min, washed once with 10 mM Tris/HCl pH 7.0, and resuspended in 2 ml of the same buffer.

Cell-free crude extract of the recombinant E. coli which comprised the dehydrogenase genes was obtained by cell disruption with 0.7 ml of glass beads (d=0.5 mm) in a vibratory mill (3×5 min with intermediate cooling on ice).

The consumption of reduced cosubstrates can be followed during the reduction of ketones in a photometer at 340 nm. 10 μl of diluted cell-free crude extract (≅10 μg of protein), 10 μmol of ketone and 250 nmol of NADH or NADPH were incubated in 1 ml of 50 mM KPᵢ, 1 mM MgCl₂, pH 6.5, at 30° C. 1 Unit (1 U) corresponds to the amount of enzyme which reduces 1 μmol of ketone in 1 min.

### Example 5

### Preparation of (S)-1-pyridin-4-ylethanol on a 4 l laboratory scale

Batch-wise operation rather than metered addition was used for the pyridin-4-ylethanone since this simplified handling in the pilot plant. Furthermore, the solvent used was 2-butanol rather than isopropanol. In a further experiment, the amount of isopropanol was reduced from 40% to 20% to reduce the subsequent distillation time.

To this end, 0.8 l of isopropanol or 2 l of 2-butanol in a 33 mM KH₂PO₄ buffer (pH 6.5) without added MgCl₂ or MgSO₄ (this may result in poisoning of the catalyst during the subsequent hydrogenation of the ring) were initially charged in a heatable 4 l reactor with stirrer. 0.5 g (0.2 mM) of NAD and 483 g (1M) of pyridin-4-ylethanone were added. The reaction was started by addition of 100 ml of the biocatalyst LU11558 in the form of whole cells (untreated fermenter discharge). The single-phase reaction mixture was at 40° C. The pH remained constant at pH-6.5. Every hour, a sample was removed, quenched with conc. HCl and analyzed by HPLC (GV31366/132). The total volume of the batch was 4 l.

After about 24 h, the reaction mixture was removed and worked up.

Batchwise operation with 50% 2-butanol (v/v) showed virtually complete conversion after 5 hours. About 1.6% ketone remain; this can be reduced to 1.4% overnight.

However, one problem of this operation is the work-up that follows: Only one phase is obtained, not a two-phase mixture as expected. This is presumably due to the large amounts of pyridin-4-ylethanone, which may act as a solubilizer. Even when water or 2-butanol are added to the discharge, there is no phase separation, and a distillation is therefore required.

In the batch-wise reaction with 20% isopropanol, the reduction was considerably slower compared to 2-butanol. After about 24 h, there was virtually complete conversion with about 1.9% of ketone remaining.

However, with regard to work-up, the use of isopropanol instead of 2-butanol has advantages since isopropanol is easier to remove by distillation. Moreover, by reducing the volume of isopropanol, the distillation time is reduced.

### Work-up:

The reduction discharges are initially in each case filtered through Celite®.

When using 50% 2-butanol as auxiliary alcohol, a single-phase reaction mixture is obtained which, on concentration on a rotary evaporator, does not give a clear, fast phase separation. By adding 10% by weight of NaCl, a good fast phase separation with a crud layer concentrated at the separation zone is obtained. Concentration of the organic phase on a rotary evaporator affords the desired product in a yield of 82%. The chlorine content is 0.1% and will presumably act as a catalyst poison during subsequent hydrogenation. This was also the reason to dispense with any chloride-containing salts (e.g. MgCl₂) during the enzymatic reduction (see 2.2). Counter-extraction of the organic phases with water resulted in a significant loss of product owing to the good solubility of the (S)-1-pyridin-4-ylethanol in water.

The procedure with 20% isopropanol (Raschig ware) was found to be the method of choice for the pilot plant phase owing to the simple work-up: after filtration through Celite®, the single-phase mixture is initially concentrated to about 50% (w/w) at 50-60° C. on a rotary evaporator.

A first extraction is then carried out using double the amount (w/w) of ethyl acetate either at room temperature or at 50° C. to achieve a more rapid phase separation. In each case, a crud layer is formed between aqueous and organic phase. The crud layer may be separated off with the organic (a) or with the aqueous phase (b). In case a, another filtration is required. Removal of the solvent on a rotary evaporator gives 78% of the desired product as a light-sandcolored solid. Another extraction of the aqueous phase with 150% (w/w) ethyl acetate and work-up according to (a) gives a further 10%, i.e. 88% yield in total, of (S)-1-pyridin-4-ylethanol in the 4 l laboratory scale (ee>99%, chem. purity>98%).

### Example 6

### Preparation of (S)-1-piperidin-4-ylethanol

In a first step, a customary ring hydrogenation catalyst was tested for the intended reaction. This was a supported metal catalyst consisting of 5% Ru on Al₂O₃. These contacts are intended for a fixed bed and were therefore ground for batch-wise operation.

What was reacted was a 26% strength solution of enantiomerically pure (S)-1-pyridin-4-yl-ethanol from the enzymatic reduction of 4-acetylpyridine, Example 5) in methanol at 130° C. and 200 bar H₂ with a catalyst load of 1.5% by weight based on the starting material.

The catalyst tested showed very good conversion combined with likewise very good selectivity, it was therefore used for the optimization work that followed.

The conversion was >99%, the selectivity was 93%. The ee was 96%.

### Further Optical Enrichment

The ee can be increased even more by precipitating the crude material with (R)-mandelic acid.

To this end, an equimolar amount of (R)-mandelic acid was added to the alcohol in isopropanol, and the mixture was heated to reflux. When the mixture was cooled slowly, individual crystals began to form from 73° C. onwards. Using a ramp of −5° C./h, the temperature was reduced to 20° C. and the crystals were filtered off. The solids loading was about 11% and the filtration resistance was 1.36*10¹² meas/m².

The desired alcohol was released from the mandelic acid salt. The ee could be increased to 99.2%, at a total yield of 76%. The chemical purity was >99.5%.

1. A process for preparing N-heterocyclic optically active alcohols of the formula I 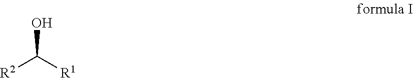
in which
R¹ denotes alkyl groups which for their part may be mono- or polysubstituted by alkyl, halogen, SH, SR³, OH, OR³, NO₂, CN, CO, COOR³, NR³R⁴ or NR³R³R⁵⁺X⁻, where R³, R⁴ and R⁵ independently of one another are H or a lower alkyl or lower alkoxy radical and X⁻ is a counterion
R² denotes N-containing heteroaryl groups which for their part may be mono- or poly-substituted by alkyl, halogen, SH, SR³, OH, OR³, NO₂, CN, CO, COOR³, NR³R⁴ or NR³R³R⁵⁺X⁻, where R³, R⁴ and R⁵ independently of one another are H or a lower alkyl or lower alkoxy radical and X⁻ is a counterion
by reduction of the corresponding ketone, where the reduction is carried out using a dehydrogenase having the polypeptide sequence SEQ ID NO:2 or NO:4, or using a polypeptide sequence where, compared to SEQ ID NO:2 or NO:4, up to 25% of the amino acid residues are modified by deletion; insertion, substitution or a combination thereof.

2. The use of a process according to claim 1 in a reaction for preparing N-heterocyclic optically active alcohols of the formula III 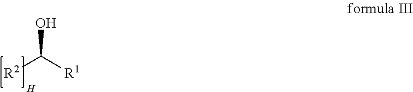
where the alcohol of the formula I obtained according to claim 1 is reacted further by hydrogenation of the N-containing heteroaryl radical R².

3. A process for preparing N-heterocyclic optically active alcohols of the formula III 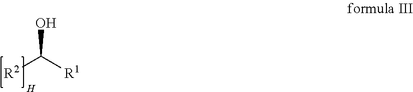
by carrying out the process according to claim 1 in a first step (a) and converting, in a second step (b), the optically active alcohol of the formula I obtained in (a) into III by hydrogenation.

4. The process according to claim 3 wherein the hydrogenation in step (b) is carried out using a ruthenium catalyst on Al₂O₃.

5. The process according to claim 1 wherein R² is 4-pyridinyl and R¹ is methyl.
